(19) United States
(12) Patent Application Publication
(10) Pub. No.: US 2018/0155263 A1
(43) Pub. Date: Jun. 7, 2018

(54) METHODS AND CATALYSTS FOR CONVERTING METHANE TO METHANOL

(71) Applicant: NOVOFORM TECHNOLOGIES LLC, Los Angeles, CA (US)

(72) Inventor: Ramez ELGAMMAL, Los Angeles, CA (US)

(21) Appl. No.: 15/512,012

(22) PCT Filed: Sep. 17, 2014
(86) PCT No.: PCT/US14/56111

§ 371 Date: Mar. 16, 2017

(51) Int. Cl. C07C 29/50; B01J 23/72; B01J 23/34; B01J 23/889; B01J 21/08

(52) U.S. Cl. CPC C07C 29/50; B01J 23/72; B01J 21/08; B01J 23/8892; B01J 23/34

(57) ABSTRACT

The invention encompasses methods of directly converting methane- to methanol The invention further encompasses catalysts that efficiently afford this transformation at low temperatures. Exemplary embodiments encompassed by the invention include a gas stream containing methane gas and oxygen,—which is passed over an oxygen-activated catalyst to directly form methanol

### FIELD OF THE INVENTION

This invention generally relates to processes of directly converting methane to methanol and to catalysts that afford this transformation. Specifically, the invention encompasses low temperature methods and systems for the direct and selective transformation of methane to methanol.

### BACKGROUND OF THE INVENTION

The major component of natural gas is methane and its large abundance and the increased ability to recover it efficiently have made natural gas an important source of energy. Transportation of methane remains a challenge because under ambient temperature and pressure it exists in a gaseous state. This problem could be readily addressed by oxidizing methane to methanol, which exists as a liquid under ambient conditions. The current industrial process to manufacture methanol from methane involves an indirect, two-step process where methane is reacted with steam at high temperatures over a specified catalyst (e.g., 850 degrees Celsius) and high pressures (e.g., 10-20 atm) to produce syngas, a mixture of H₂and CO. Methanol is subsequently produced by heating syngas over a second catalyst at very high pressures (e.g., 50-100 atm).

It would be advantageous to avoid the syngas intermediate and to develop a direct method to oxidize methane to methanol. However, a direct oxidation process has proven to be challenging in part due the strong carbon-hydrogen bonds in methane and over-oxidation of methane to other oxygenated species (e.g., CO₂). Accordingly, there is a need in the art to develop new techniques and catalysts whereby direct oxidation of methane to methanol can take place to avoid the high temperatures and pressures currently required for this conversion. The present invention addresses this need.

### SUMMARY OF THE INVENTION

The invention generally encompasses methods of converting methane to one or more oxidative products, for example, but not limited to, methanol and/or dimethyl ether. In certain embodiments, the invention encompasses methods of directly converting methane to methanol. In certain embodiments, the invention encompasses methods of directly converting methane to dimethyl ether. In certain embodiments, the invention encompasses methods of directly converting methane to methanol and dimethyl ether. Furthermore, the invention provides catalysts that efficiently afford this transformation at low temperatures. The oxidizing environment may be composed of a feed of molecular oxygen or air. A gas stream containing methane is passed over the oxygen-activated catalyst to directly form methanol.

In one illustrative embodiment, the invention encompasses a catalyst comprising:

a solid matrix;

at least one transition metal;

at least one ligand covalently bound to the solid matrix, and

oxygen bound to the transition metal.

In certain exemplary embodiments, the oxygen is reversibly bound to the transition metal.

In certain exemplary embodiments, the ligand is bound to said transaction metal.

In certain exemplary embodiments, the silica matrix is mesoporous or nanoporous silica.

In certain exemplary embodiments, the transition metal is selected from the group consisting of manganese, iron, cobalt, nickel, copper, and combinations thereof.

In certain exemplary embodiments, the ligand comprises a moiety selected from an imidazole moiety, a triazole moiety, a pyrazole moiety, a pyridine moiety, and a tetrazole moiety.

In certain exemplary embodiments, the imidazole moiety, triazole moiety, pyrazole moiety, pyridine moiety, and tetrazole moiety include those depicted in FIG. 4, wherein R₁ to R₂₃ are independently selected from H, amino, alkyl, substituted alkyl, heteroalkyl, substituted heteroalkyl, cycloalkyl, substituted cycloalkyl, heterocycloalkyl, substituted heterocycloalkyl, aryl, substituted aryl, heteroaryl, substituted heteroaryl, aralkyl, substituted aralkyl, hydroxyl, alkoxy, alkenyl, substituted alkenyl, alkynyl, substitute alkynyl, amide, azo, benzyl, substituted benzyl, carbonate, acyl, carboxylate, amide, sulfonamide, cyanate, ether, ester, halide, imine, isocyanide, isocyanate, oxy, sulfonyl, nitrile, nitro, nitroso, thiol, and substituted thiol.

In another embodiment, the invention encompasses methods for synthesizing an oxygen-activated catalyst, the method comprising: (i) contacting a pre-catalyst with oxygen (calcination) in a gaseous environment, thereby forming said oxygen-activated catalyst, wherein the pre-catalyst comprises (a) a solid matrix; (b) at least one transition metal, and (c) at least one ligand covalently bound to said solid matrix.

In certain exemplary embodiments, the ligand is bound to said transition metal.

In certain exemplary embodiments, the contacting said pre-catalyst with said oxygen occurs at a temperature from about 730° C. to about 950° C.

In certain exemplary embodiments, the solid matrix is a silica matrix.

In certain exemplary embodiments, the silica matrix is mesoporous or nanoporous silica.

In certain exemplary embodiments, the method further comprises: (ii) reacting said solid matrix with a ligand precursor, thereby forming a ligand-grafted solid matrix.

In certain exemplary embodiments, the solid matrix is a mesoporous silica template selected from SBA-15 and MCM-41.

In certain exemplary embodiments, the ligand precursor comprises an imidazole moiety, a triazole moiety, a pyrazole moiety, a pyridine moiety, or a tetrazole moiety.

In certain exemplary embodiments, the ligand precursor further comprises a silyl ether moiety.

In certain exemplary embodiments, the ligand precursor has a structure according to Formula I, Formula II, Formula III, Formula IV, Formula V, Formula VI, Formula VII, Formula VIII, or Formula IX as shown in FIG. 4, wherein R₁ to R₂₃ are independently selected from H, amino, alkyl, substituted alkyl, heteroalkyl, substituted heteroalkyl, cycloalkyl, substituted cycloalkyl, heterocycloalkyl, substituted heterocyloalkyl, aryl, substituted aryl, heteroaryl, substituted heteroaryl, aralkyl, substituted aralkyl, hydroxyl, alkoxy, alkenyl, substituted alkenyl, alkynyl, substitute alkynyl, amide, azo, benzyl, substituted benzyl, carbonate, acyl, carboxylate, amide, sulfonamide, cyanate, ether, ester, halide, imine, isocyanide, isocyanate, oxy, sulfonyl, nitrile, nitro, nitroso, thiol, and substituted thiol.

In certain exemplary embodiments, the ligand precursor is selected from N-(3-propytrimethoxysilane) imidazole and N-(3-propytrimethoxysilane) 1,2,4-triazole.

In certain exemplary embodiments, the method further comprises: (iii) reacting said ligand-grafted solid matrix with a transition metal salt, thereby forming said pre-catalyst.

In certain exemplary embodiments, the transaction metal is selected from the group consisting of manganese, iron, cobalt nickel, copper, and combinations thereof.

In certain exemplary embodiments, the transaction metal is selected from the group consisting of manganese, copper, and combinations thereof.

In certain exemplary embodiments, the method further comprises (ii) reacting a ligand precursor with tetraethyl orthosilate (TEOS) at a ratio of TEOS:ligand precursor from about 4 to 24; and optionally adding a structure-direction agent, thereby forming a ligand-grafted silica matrix.

In certain exemplary embodiments, the structure-directing agent is an amine-based surfactant.

In certain exemplary embodiments, the amine-based surfactant is selected from alkyl amines, for example, n-C₁₀-C₂₀ alkyl amines, including, but not limited to, n-hexadecylamine and n-octadecylamine.

In certain exemplary embodiments, the ligand precursor comprises an imidazole moiety, a triazole moiety, a pyrazole moiety, a pyridine moiety, or a tetrazole moiety.

In certain exemplary embodiments, the ligand precursor further comprises a silyl ether moiety.

In certain exemplary embodiments, the ligand precursor has a structure according to Formula I, Formula II, Formula III, Formula IV, Formula V, Formula VI, Formula VII, Formula VIII, or Formula IX as shown in FIG. 4, wherein R₁ to R₂₃ are independently selected from H, amino, alkyl, substituted alkyl, heteroalkyl, substituted heteroalkyl, cycloalkyl, substituted cycloalkyl, heterocycloalkyl, substituted heterocyloakyl, aryl, substituted aryl, heteroaryl, substituted heteroaryl, aralkyl, substituted aralkyl, hydroxyl, alkoxy, alkenyl, substituted alkenyl, alkynyl, substitute alkynyl, amide, azo, benzyl, substituted benzyl, carbonate, acyl, carboxylate, amide, sulfonamide, cyanate, ether, ester, halide, imine, isocyanide, isocyanate, oxy, sulfonyl, nitrile nitro, nitroso, thiol, and substituted thiol.

In certain exemplary embodiments, the ligand precursor is selected from N-3-propyltrimenthoxysilane) imidazole and N-(3-propyltrimenthoxysilane) 1,2,4-triazole.

In certain exemplary embodiments, the method further comprises, reacting said ligand-grafted silica matrix with a transition metal salt, thereby forming said pre-catalyst.

In certain exemplary embodiments, the transition metal is selected from the group consisting of manganese, iron, cobalt, nickel, copper, and combinations thereof.

In certain exemplary embodiments, the transition metal is selected from the group consisting of manganese, copper, and combinations thereof.

In certain exemplary embodiments, the method further comprises silylating said pre-catalyst or said oxygen-activated catalyst thereby forming a silylated pre-catalyst or a silylated oxygen-activated catalyst.

In certain exemplary embodiments, the invention encompasses and oxygen-activated catalyst made according to a method disclosed therein.

In certain exemplary embodiments, the invention encompasses a method for directly converting methane (CH₄) to methanol (CH₃—OH) comprising, contacting a gas feed comprising methane with an oxygen-activated catalyst under conditions sufficient to form said methanol.

In certain exemplary embodiments, the gas feed is contacted with said oxygen-activated catalyst at a temperature below about 750° C.

In certain exemplary embodiments, the gas feed is contacted with said oxygen-activated catalyst at a temperature from about 350° C. to about 600° C.

In certain exemplary embodiments, the gas feed is contacted with said oxygen-activated catalyst at a temperature from about 150° C. to about 350° C.

In certain exemplary embodiments, the gas feed is contacted with said oxygen-activated catalyst at a pressure of less than about 50 atm.

In certain exemplary embodiments, the gas feed is contacted with said oxygen-activated catalyst at a pressure of less than about 20 atm.

In certain exemplary embodiments, the gas feed is contacted with said oxygen-activated catalyst at ambient (atmospheric) pressure.

In certain exemplary embodiments, the gas feed further comprises oxygen.

In certain exemplary embodiments, the gas feed further comprises a carrier gas.

In certain exemplary embodiments, the method further comprises collecting said methanol.

In certain exemplary embodiments, the invention encompasses a method for directly converting methane to methanol at a temperature of less than 750° C., said method comprising: contacting a gas feed comprising methane with an oxygen-activated catalyst, thereby forming said methanol from said methane, wherein said oxygen-activated catalyst comprises:

a solid matrix;

at least one transition metal;

at least one ligand covalently bound to said solid matrix; and

oxygen bound to said transition metal.

In certain exemplary embodiments, the oxygen is reversibly bound to the transition metal.

In certain exemplary embodiments, the ligand is bound to said transition metal.

In certain exemplary embodiments, the solid matrix is a silica matrix.

In certain exemplary embodiments, the silica matrix is mesoporous or nanoporous silica.

In certain exemplary embodiments, the transition metal is selected from the group consisting of manganese, iron, cobalt, nickel, copper, and combinations thereof.

In certain exemplary embodiments, the transition metal is selected from the group consisting of manganese, copper, and combinations thereof.

In certain exemplary embodiments, the ligand comprises a moiety selected from am imidazole moiety, a triazole moiety, a pyrazole moiety, a pyridine moiety, and a tetrazole moiety.

In certain exemplary embodiments, the imidazole moiety, triazole moiety, pyrazole moiety, pyridine moiety, and tetrazole moiety are selected from those depicted in FIG. 4, wherein R₁ to R₂₃ are independently selected from H, amino, alkyl, substituted alkyl, heteroalkyl, substituted heteroalkyl, cycloalkyl, substitute cycloalkyl, heterocycloalkyl, substituted heterocycloalkyl, aryl, substituted aryl, heteroaryl, substituted heteroaryl, aralkyl, substituted aralkyl, hydroxyl, alkoxy, alkenyl, substituted alkenyl, alkynyl, substitute alkynyl, amide, azo, benzyl, substituted benzyl carbonate, acyl, carboxylate, amide, sulfonamide, cyanate, ether, ester, halide, imine, isocyanide, isocyanate, oxy, sulfonyl, nitrile, nitro, nitroso, thiol, and substituted thiol.

In certain exemplary embodiments, the gas feed is contacted with said oxygen-activated catalyst at a pressure of less than about 50 atm.

In certain exemplary embodiments, the gas feed is contacted with said oxygen-activated catalyst at a pressure of less than about 2 atm.

In certain exemplary embodiments, the pressure is ambient (atmospheric) pressure.

In certain exemplary embodiments, the invention encompasses an apparatus for the direct conversion of methane gas to methanol comprising:

A storage unit for methane gas;

A contacting unit for passing a gas feed comprising methane gas and oxygen over an oxygen-activated catalyst.

In certain exemplary embodiments, the apparatus further comprises a collecting unit for removing methanol from said contacting unit.

In certain exemplary embodiments, the apparatus further comprises a heating unit for heating said oxygen-activated catalyst to a temperature of less than 750° C.

### Reaction Temperature

The catalyst may be heated directly by an external source or by a heated stream of methane and the oxygen containing gas stream. The temperature at which the reaction occurs is less than 850 degrees Celsius (° C.), e.g., less that 750° C., less than 700° C., less than 600° C., less than 500° C., less than 400° C., less than 300° C., or less than 200° C. In other examples, the temperature is in a temperature range of about 150 degrees Celsius to about 350 degrees Celsius. In other examples the temperature range is from about 350 degrees Celsius to about 500 degrees Celsius. In further examples the temperature range is about 500 degrees Celsius to 650 degrees Celsius. In further examples the temperature range is about 600 degrees Celsius to 750 degrees Celsius. In further examples the temperature range is about 700 degrees Celsius to 850 degrees Celsius. In other examples, the temperature in in a temperature range from about 100° C., from about 1000° C., from about 100° C. to about 900° C., from about 100° C. to about 800° C., from about 100° C. to about 700° C., from about 100° C. to about 600° C., from about 100° C. to about 500° C., from about 100° C. to about 400° C., or from about 100° C. to about 300° C. In other examples, the temperature is from about 150° C. to about 900° C., from about 150°C. to about 800° C., from about 150° C. to about 700° C., from about 150° C. to about 600° C., from about 150° C. to about 500° C., from about 150° C. to about 400° C., or from about 150° C. to about 300° C. In other examples the reaction temperature is from about 200° C. to about 900° C., from about 200° C. to about 800° C., from about 200° C. to about 700° C., from about 200° C. to about 600° C., from about 200° C. to about 500° C., from about 200° C. to about 400° C., or from about 200° C. to about 300° C. In other examples the reaction temperature is from about 300° C. to about 1000° C., from about 300° C. to about 900° C., from about 300° C. to about 800° C., from about 300° C. to about 700° C., from about 300° C. to about 600° C., from about 300° C. to about 500° C., from about 300° C. to about 400° C. In some examples, the temperature is from about 250° C. to about 300° C. In other examples, the temperature is from about 400° C. to about 700° C., from about 400° C. to about 600° C., or from about 400° C. to about 500° C.

### Gas Feed

The total pressure of the gas feed in the reaction is topically less than 100 atm. In some examples, the pressure is less than 80 atm, less than 60 atm, less than 50 atm, less than 40 atm, less than 30 atm, less than 20 atm, or less than 10 atm. In other examples, the catalyst is contacted with the gas feed at a pressure from about 1 atm to about 100 atm, from about 1 atm to about 80 atm, from about 1 atm to about 60 atm, from about 1 atm to about 50 atm, from about 1 atm to about 40 atm, from about 1 atm to about 30 atm, or from about 1 atm to about 20 atm. In other examples, the catalyst is contacted with the gas feed at a pressure from about 2 atm to about 100 atm, from about 2 atm to about 80 atm, from about 2 atm to about 60 atm, from about 2 atm to about 50 atm, from about 2 atm to about 40 atm, from about 2 atm to about 30 atm, or from about 2 atm to about 20 atm. In some examples, the pressure is from about 2 atm to about 15 atm.

In other examples, the gas feed is contacted with the catalyst at ambient (atmospheric) pressure. In certain embodiments, the gas feed contains only methane. In certain embodiments, the gas feed contains not only methane. In some embodiments, the gas feed further includes oxygen. The gas feed can contain oxygen gas, or may contain air. The gas feed may also contain a carrier gas (e.g., non-reactive gas), examples of which include, but are not limited to, helium and/or nitrogen. In some examples, the gas feed is substantially free of syngas (i.e., a mixture containing hydrogen gas and carbon monoxide).

### Oxygen Activated Catalysts

The invention further includes oxygen-activated catalysts that afford the direct conversion of methane to one or more oxidative products, for example, but not limited to methanol and/or dimethyl ether. The invention further includes oxygen-activated catalysts that selectively afford the direct conversion of methane to methanol. The invention further includes oxygen-activated catalysts that selectively afford the direct conversion of methane to dimethyl ether. In certain embodiments, the oxygen-activated catalysts involves a series of chemical transformation. First, a pre-catalyst is synthesized. In certain embodiments, the pre-catalysts are, for example, functionalized mesoporous or nanoporous silica materials that contain ligands in the pores or on the surface. In certain embodiments, where the ligands reside in the pores, a common method to sythesize these materials is by self-assembly using a templating agent. In certain embodiments, this strategy involves co-hydrolysis and polycondensation reactions. In one illustrative example, the catalysts synthesized by self-assembly may contain a worm-hole like structure. In another illustrative example, the self-assembled pre-catalysts may also be crystallographically disordered. In an additional illustrative example, the self-assembled catalysts may be amorphous. In a further example, the self-assembled pre-catalysts may contain an ordered structure, one illustrative such example being hexagonal. The size of the pores and their morphologies are controlled by, but not limited to, for example, the synthesis conditions including temperature, concentration, specific reagents, and templating agents. Additionally, the pre-catalysts may be synthesized using, for example, post-synthetic grafting. In certain embodiments, post-synthetic grafting begins with a preordered silica template, which includes but is not limited to, for example, SBA-15 and MCM-41. In certain illustrative embodiments, a ligand is then reacted with a silicon-OH bone. In certain embodiments, both the self-assembled and post-synthetic grafted pre-catalyst are impregnated with a transition metal forming a covalent or ionic interaction with the ligands and/or silica framework. One illustrative method of preparing these species is a solvothermal reaction of a transition metal salt and the pre-catalyst.

In certain embodiments, the oxygen-activated catalyst is then formed by calcination or heating the metal impregnated pre-catalyst in the presence of molecular oxygen. A temperature range of about 370 degrees Celsius to about 750 degrees Celsius and at ambient pressure (preferably about 400 degrees Celsius to about 600 degrees Celsius in a continuous gas flow) is typically used to form the oxygen-activated catalysts.

The invention also provides method of creating an oxygen-activated catalyst suitable for direct conversion of methane to methanol at ambient a pressure. In this method a catalyst is pre-treated by heating the catalyst in a gaseous environment with continuous gas flow and at a pre-treatment temperature range of about 370 degrees Celsius to about 950 degrees Celsius to form an oxygen-activated catalyst.

### Apparatus/Processing Plant

The invention further encompasses an apparatus (e.g., a chemical processing plant) for direct conversion of methane to methanol. The apparatus includes a storage unit for methane gas, a storage unit for an oxygen-activated catalyst and a contacting unit for passing the methane gas over the oxygen-activated catalyst from the respective storage units, e.g., at a temperature of less than 750 degrees Celsius with an oxygen-containing gas feed for the direct conversion of methane gas into methanol. In certain embodiments, the plant could further include a collecting unit for removing the methanol from the contacting unit. In some embodiments, the invention encompasses an apparatus for direct conversion of methane to methanol comprising or substantially consisting of: (a) a storage unit for methane gas, (b) a storage unit for an oxygen-activated catalyst according to the invention, (c) a contacting unit for passing a gas feed containing methane over the oxygen-activated catalyst, e.g., at a temperature of less than 750 degrees Celsius to form methanol, (d) optionally a storage unit for oxygen gas, and (e) optionally a collecting unit for removing methanol from the contacting unit. In some examples according to any of the above embodiments, the gas feed includes oxygen.

### BRIEF DESCRIPTION OF THE DRAWINGS

FIG. 1 is an exemplary illustration of the process steps involved in the direct selective conversion of methane to methanol according to an embodiment of the invention.

FIG. 2 is an exemplary illustration of schematically the synthetic steps to produce an exemplary oxygen-activated post-synthetic grafter catalyst beginning with a mesoporous silica scaffold, e.g., SBA-15, MCM-41, etc.

FIG. 3 is an exemplary illustration of schematically synthetic steps to produce oxygen-activated self-assembled catalysts of the invention.

FIG. 4 illustrates exemplary ligands for both the post-synthetic grafted and self-assembled catalysts of the invention.

FIG. 5 illustrates exemplary metal salts that could be used to impregnate the pre-catalysts.

FIG. 6 illustrates exemplary post-synthetically grafted pre-catalysts comprising more than one metal.

FIG. 7 illustrates exemplary post-synthetically grafted pre-catalysts comprising more than one metal and more than one ligand type.

FIG. 8 illustrates exemplary self-assembled pre-catalysts comprising more than one metal and more than one ligand type.

FIG. 9 illustrates exemplary methods to silylate the surface of the catalysts.

### DETAILED DESCRIPTION OF THE INVENTION

The invention generally encompasses methods of converting methane to one or more oxidative products, for example, but not limited to, methanol and/or dimethyl ether. In certain embodiments, the invention encompasses methods of directly converting methane to methanol. In certain embodiments, the invention encompasses methods of directly converting methane to dimethyl ether. In certain embodiments, the invention encompasses methods of directly converting methane to methanol and dimethyl ether. The following scheme illustrates the general nature of the reaction encompassed by the invention.

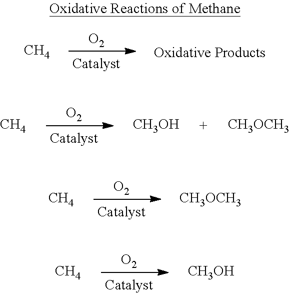

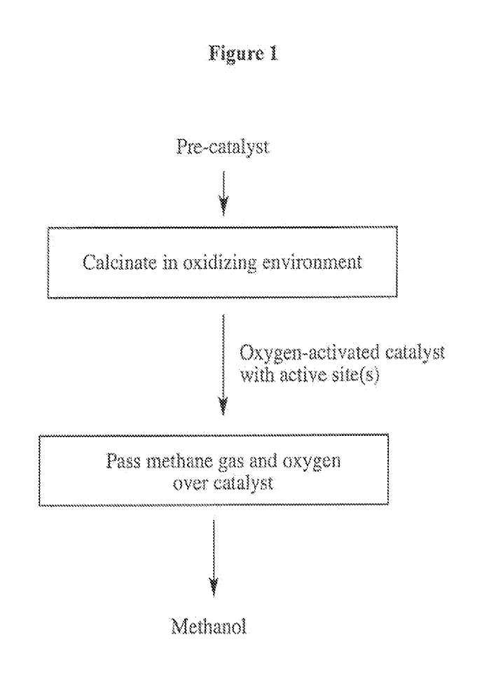

In an exemplary embodiment, the invention encompasses a process for the direct and selective oxidation of methane to methanol at low temperature. FIG. 1 illustrates an exemplary process of the invention. The exemplary process involves the formation of a pre-catalyst, which is heated in an oxidizing atmosphere to form an oxygen-activated catalyst. This leads to the formation of an active site in the oxygen-activated catalyst, which facilitates the direct conversion of methane to methanol. Next, methane gas is contacted with or passed over the oxygen-activated catalyst to directly form methanol. The entire reaction (i.e., creation of the active site and passing methane gas) is carried out at temperatures, for example, below 750 degrees Celsius and at ambient pressure. Finally, methanol is collected from the reaction vessel.

In another example, a gas stream containing methane is contacted with or passed over the oxygen-activated catalyst to directly form methanol. The catalyst may be heated directly by an external source or by a heated stream of methane and the oxygen containing gas stream. In certain embodiments, the temperature of the reaction is less than 750 degrees Celsius. In other examples the temperature could be in a temperature range of about 150 degrees Celsius to about 350 degrees Celsius. In other examples the temperature range may be about 350 degrees Celsius to about 500 degrees Celsius. In further examples the temperature range may about 500 degrees Celsius to about 750 degrees Celsius. In certain embodiments, the total pressure of the gas feed in the reaction is typically less than 50 atm. This gas feed is composed of methane and oxygen and/or may contain air. In certain embodiments, the gas feed may also be partially composed of a carrier gas, examples of which may include, for example, helium and/or nitrogen.

In another exemplary embodiment, the invention encompasses a process for the direct and selective oxidation of methane to dimethyl ether at low temperatures. FIG. 1 illustrates an exemplary process of the invention. The exemplary process involves the formation of a pre-catalyst, which is heated in an oxidizing atmosphere to form a oxygen n-activated catalyst. This leads to the formation of an active site in the oxygen-activated catalyst, which facilitates the direct conversion of methane to dimethyl ether. Next, methane gas is contacted with or passed over the oxygen-activated catalyst to directly form dimethyl ether. The entire reaction (i.e., creation of the active site and passing methane gas) is carried out at temperatures, for example, below 750 degrees Celsius and at ambient pressure. Finally, dimethyl ether is collected from the reaction vessel.

In another example, a gas stream containing methane is contacted with or passed over the oxygen-activated catalyst to directly form dimethyl ether. The catalyst may be heated directly by an external source or by a heated stream of methane and the oxygen containing gas stream. In certain embodiments, the temperature of the reaction is less than 750 degrees Celsius. In other examples the temperature could be in a temperature range of about 150 degrees Celsius to about 350 degrees Celsius. In other examples the temperature range may be about 350 degrees Celsius to about 500 degrees Celsius. In further examples the temperature range may about 500 degrees Celsius to about 750 degrees Celsius. In certain embodiments, the total pressure of the gas feed in the reaction is typically less than 50 atm. This gas feed is composed of methane and oxygen and/or may contain air. In certain embodiments, the gas feed may also be partially composed of a carrier gas, examples of which may include, for example, helium and/or nitrogen.

### Definitions

The definitions and explanations below are for the terms as used throughout this entire document including both the specification and the claims. Throughout the specification and the appended claims, a given formula or name shall encompass all isomers thereof, such as stereoisomers, geometrical isomers, optical isomers, tautomers, and mixtures thereof where such isomers exist.

The term “direct”or “directly” in the context of methane conversion to methanol refers to a process, in which no substantial amount of an intermediate (e.g., gaseous intermediate), such as hydrogen gas (H₂) and/or carbon monoxide (CO) is formed and/or isolated. In some examples, the process does not involve the formation of syngas. In one example, the process is a one-step process. In certain exemplary embodiments, the process of “directly” converting methane to methanol doe snot involve substantial formation of oxygenated species other than methanol. For example, the “direct” process does not involve the substantial formation of carbon dioxide (CO₂).

The terms “oxidative product(s)” or “oxygenated species” refers to any products that result from the oxidation of methane using the methods disclosed herein. Oxidative products as used therein include methanol, dimethyl ether, formaldehyde, formic acid, etc. Preferably, oxidative products as used herein include methanol and dimethyl ether. More preferably, oxidative product as used herein include only methanol.

The term “bound” or “bound to” (or any grammatical variation thereof) in the context of chemical structure refers to various types of chemical bonds, such as covalent bonds (e.g., non-polar and polar), coordinate covalent (i.e., dipolar bonds), ionic bonds, metallic bonds, bonds with covalent as well as ionic character, metallic coordination (i.e., coordination complex or metal complex). In certain illustrative embodiments, the term “bound” or “bound to” refers to a chemical bond forming a metal complex or coordination complex. In some examples, the transition metal contained in the catalysts of the invention is (e.g., reversibly or irrepressibly) coordinated to oxygen. In other examples, the transition metal can be coordinated to hydroxyl groups located on a solid matrix, such as a silica matrix. In other examples, ligands, which are covalently bound to the surface of a solid matrix (e.g., a silica matrix), are additionally bound to a transition metal forming a ligand-metal complex (coordination complex). In other illustrative embodiments, a multitude of bonds formed between oxygen and the transition metal (e.g., during calcination of the catalyst), or between oxygen, ligands, and the transition metal create catalytic sites capable of catalyzing the conversion of methane to methanol (e.g., under reaction conditions described herein).

The term “ligand” refers to a chemical moiety comprising at least one heteroatom. In some embodiments, a ligand comprises a heterocyclic or heteroaryl moiety. In other examples, the ligand is capable of forming a ligand transition metal complex.

The terms “solid matrix,” “template,” or “substrate” means a solid carrier material. In some examples, the solid matrix has a large surface area (e.g., is a porous material). In other examples, the solid matrix has functional groups (e.g., hydroxyl groups), which can be used to form a covalent bond to a ligand. In some examples, the solid matrix is a silica matrix (e.g., mesoporous or nanoporous silica).

The term “transition metal” is used within its art-recognized meaning. For example, a transition metal is an element is an element whose atom has a partially filled d sub-shell, or which can give rise to cations with an incomplete d sub-shell. In other examples, the transition metal is selected from elements found in groups 3 to 12 of the periodic table and f-block lanthanides and actinides.

The terms “alkyl” by itself for as part of another substituent, means, unless otherwise stated, a straight or branched chain hydrocarbon radical having the number of carbon atoms designated (e.g., C₁-C₁₀ means one to ten carbon atoms). Typically, an alkyl group will have from 1 to 24 carbon atoms, for example having from 1 to 10 carbon atoms, from 1 to 8 carbon atoms or from 1 to 6 carbon atoms. A “lower alkyl” group is an alkyl group having from 1 to 4 carbon atoms. The term “alkyl” includes di- and multivalent radicals. For example, the terms “alkyl” includes “alkylene” wherever appropriate, e.g., when the formula indicates that the alkyl group is divalent or when substituents are joined to form a ring. Examples of alkyl radicals include, but are not limited to, methyl, ethyl, n-propyl, iso-propyl, n-butyl, tert-butyl, iso-butyl, sec-butyl, as well as homologs and isomers of, for example, n-pentyl, n-hexyl, n-heptyl and n-octyl.

The term “alkylene” by itself or as part of another substituent means a divalent (diradical) alkyl group, wherein alkyl is defined herein. “Alkylene”is exemplified, but not limited by —CH₂CH₂CH₂CH₂—. Typically, an “alkylene” group will have from 1 to 24 carbon atoms, for example, having 10 or fewer carbon atoms (e.g., 1 to 8 or 1 to 6 carbon atoms). A “lower alkylene” group is an alkylene group having from 1 to 4 carbon atoms.

The term “alkenyl” by itself or as part of another substituent refers to a straight or branched chain hydrocarbon radical having from 2 to 24 carbon atoms and at least one double bond. A typical alkenyl group has from 2 to 10 carbon atoms and at least one double bond. In one embodiment, alkenyl groups have from 2 to 8 carbon atoms or from 2 to 6 carbon atoms and from 1 to 3 double bonds. Exemplary alkenyl groups include vinyl, 2-propenyl, 1-but-3-enyl, crotyl, 2-(butadienyl), 2,4-pentadienyl, 3-(1,4-pentadienyl), 2-isopentenyl, 1-pent-3-enyl, 1-hex-5-enyl and the like.

The term “alkynyl” by itself or as part of another substituent refers to a straight or branched chain, unsaturated or polyunsaturated hydrocarbon radical having from 2 to 24 carbon atoms and at least one triple bond. A typical “alkynyl” group has from 2 to 10 carbon atoms and at least one triple bond. In one aspect of the disclosure, alkynyl groups have from 2 to 6 carbon atoms and at least one triple bond. Exemplary alkynyl groups include prop-1 ynyl, prop-2-ynyl (i.e., propargyl), ethynyl and 3-butynyl.

The terms “alkoxy,” “alkylamino” and “alkylthio” (or thioalkoxy) are used in their conventional sense, and refer to alkyl groups that are attached to the remainder of the molecule via an oxygen atom, an amino group, or a sulfur atom, respectively.

The term “heteroalkyl,” by itself or in combination with another term, means a stable, straight or branched chain hydrocarbon radical consisting of the stated number of carbon atoms (e.g., C₂-C₁, or C₂-C₈) and at least one heteroatom chosen, e.g., from N, O, S, Si, B and P (in one embodiment, N, O and S), wherein the nitrogen, sulfur and phosphorus atoms are optionally oxidized, and the nitrogen atoms(s) are optionally quaternized. The heteroatoms(s) is/are placed at any interior position of the heteroalkyl group. Examples of heteroalkyl groups include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂-CH₂—S(O)₂—CH₃, —CH═CH—O—CH₃, —CH₂—Si(CH₃)₃, —CH₂—CH═N—OCH₃, and —CH═CH—N(CH₃)—CH₃. Up to two heteroatoms can be consecutive, such as, for example, —CH₂—NH—OCH₃ and —CH—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. Typically, a heteroalkyl group will have from 3 to 24 atoms (carbon and heteroatoms, excluding hydrogen) (3- to 24-membered heteroalkyl). In another example, the heteroalkyl; group has a total of 3 to 10 atoms (3- to 10-membered heteroalkyl) or from 3 to 8 atoms (3- to 8-membered heteroalkyl). The “heteroalkyl” includes “heteroalkylene” wherever appropriate, e.g., when the formula indicates that the heteroalkyl group is divalent or when substituents are joined to form a ring.

The term “cycloalkyl” by itself or in combination with other terms, represents a saturated or unsaturated, non-aromatic carbocyclic radical having from 3 to 24 carbon atoms, for example, having from 3 to 12 carbon atoms (e.g., C₃-C₈ cycloalkyl or C₃-C₆ cycloalkyl). Examples of cycloalkyl include, but are not limited to, cyclopropyl, cyclobutyl, cyclopentyl, cyclohexyl, cycloheptyl, 1-cyclohexenyl, 3-cyclohexenyl, cycloheptyl and the like. The terms “cycloalkyl” also includes bridged, polycyclic (e.g., bicyclic) structures, such as norbornyl, adamantyl and bicyclo[2.2.1]heptyl. The “cycloalkyl” group can be fused to at least one (e.g., 1 to 3 ) other ring selected from aryl (e.g., phenyl), heteroaryl (e.g., pyridyl) and one-aromatic (e.g., carbocyclic or heterocyclic) rings. When the “cycloalkyl” group includes a fused aryl, heteroaryl or heterocyclic ring, then the “cycloalkyl” group is attached to the remainder of the molecule via the carbocyclic ring.

The terms “heterocycloalkyl”, “heterocyclic”, “heterocycle”, or “heterocyclyl”, by itself or in combination with other terms, represents a carbocyclic, non-aromatic ring (e.g., 3- to 8-membered ring and for example 4-, 5-, 6- or 7-membered ring) containing at least one and up to 5 heteroatoms selected from, e.g., N, O, S, Si, B and P (for example, N, O and S), wherein the nitrogen, sulfur and phosphorus atoms are optionally oxidized, and the nitrogen atom(s) are optionally quaternized (e.g., from 1 to 4 heteroatoms selected from nitrogen, oxygen and sulfur), or a fused ring system of 4- to 8-membered rings, containing at least one and up to 10 heteroatoms (e.g., from 1 to 5 heteroatoms selected from N, O and S) in stable combinations known to those of skill in the art. Exemplary heterocycloalkyl groups include a fused phenyl ring. When the “heterocyclic” group includes a fused aryl, heteroaryl or cycloalkyl ring, then the “heterocyclic” group is attached to the remainder of the molecule via a heterocycle. A heteroatom can occupy the position at which the heterocycle is attached to the remainder of the molecule. Exemplary heterocycloalkyl or heterocyclic groups of the present disclosure include morpholinyl, thiomorpholinyl, thiomorpholinyl S-oxide, thiomorpholinyl S,S-dioxide, piperazinyl, homopiperazinyl, pyrrolidinyl, pyrrolinyl, imidazolidinyl, tetrahydropyranyl, piperazinyl, homopiperazinyl, pyrrolidinyl, pyrrolinyl, imidazolidinyl, tetrahydropyranyl, piperidinyl, tetrahydrofuranyl, tetrahydrothienyl, piperindinyl, homopiperidinyl, homomorpholinyl, homothiomorpholinyl, homothiomorpholinyl S,S-dioxides, ozazolidinonyl, dihydropyrazolyl, dihydropyrrolyl, dihydropyrazolyl, dihydropyridyl, didhydropyrimidinyl, dihydrofuryl, dihydropyranyl, tetrahydrothienyl S-oxide, tetrahydrothienyl S,S-dioxide, homothiomorpholinyl S-oxide, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2yl, tetrahydrothien-3-yl, 1-piperazinyl, 2-piperazinyl, and the like.

By “aryl” is meant a 5-, 6- or 7-membered, aromatic carbocyclic group having a single ring (e.g., phenyl) or being fused to other aromatic or non-aromatic rings (e.g., from 1 to 3 other rings). When the “aryl” group includes a non-aromatic ring (such as in 1,2,3,4-tetrahydronaphthyl) or heteroaryl group then the “aryl” group is bonded to the remainder of the molecule via an aryl ring (e.g., a phenyl ring). The aryl group is optionally substituted (e.g., with 1 to 5 substituents described herein). In one example, the aryl group has from 6 to 10 carbon atoms. Non-limiting examples of aryl groups include phenyl, 1-naphthyl, 2-naphthyl, quinoline, indanyl, indenyl, dihydronaphthyl, fluorenyl, tetralinyl, benzo[d][1,3]dioxolyl or 6,7,8,9-tetrahydro-5H benzo[a]cycloehptenyl. In one embodiment, the aryl group is selected from phenyl, benzo[d][1,3]dioxolyl and naphthyl. The aryl group, in yet another embodiment, is phenyl.

The term “arylalkyl” is meant to include those radicals in which an aryl group or heteroaryl group is attached to an alkyl group to create the radicals -alkyl-aryl and -alkyl-heteroaryl, wherein alkyl, aryl and heteroaryl are defined herein. Exemplary “arylalkyl” groups include benzyl, phenethyl, pryidylmethyl and the like.

By “aryloxy” is meant the group —O-aryl, where aryl is as defined herein. In one example, the aryl portion of the aryloxy group is phenyl or naphthyl. The aryl portion of the aryloxy group, in one embodiment, is phenyl.

The term “heteroaryl” or “heteroaromatic” refers to a polyunsaturated, 5-, 6- or 7-membered aromatic moiety containing at least one heteroatom (e.g., 1 to 5 heteroatoms, such as 1-3 heteroatoms) selected from N, O, S, Si and B (for example, N, O and S), wherein the nitrogen and sulfur atoms are optionally oxidized, and the nitrogen atoms(s) are optionally quaternized. The “heteroaryl” group can be a single ring or be fused to other aryl, heteroaryl, cycloalkyl or heterocycloakyl rings (e.g., from 1 to 3 other rings). When the “heteroaryl” group includes a fused aryl, cycloalkyl or heterocycloalkyl ring, then the “heteroaryl” group is attached to the remainder of the molecule via the heteroaryl ring. A heteroaryl group can be attached to the remainder of the molecule through a carbon- or heteroatom. In one example, the heteroaryl group has from 4 to 10 carbon atoms and from 1 to 5 heteroatoms selected from O, S and N. Non-limiting examples of heteroaryl groups include pyridyl, pyrimidine, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimindazolyl, benzofuranyl, furanyl, thienyl, pyrrolyl, oxadiazolyl, thiadiazolyl, triazolyl, tetrazolyl, isothiazolyl, naphthyridinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridyl, benzotetrahydrofuranyl, benzotetrahydrothienyl, purinyl, benzodioxolyl, triazinyl, pteridinyl, benzothiazolyl, imidazopyridyl, imidazothiazolyl, dihydorbenzisoxazinyl, benzisoxazinyl, benzoxazinyl, dihydrobenzisothiazinyl, benzopyranyl, benzothiopyranyl, chromonyl, chromanonyl, pyridyl-N-oxide, tetrahydroquinolinyl, dihydroquinolinyl, dihydroquinolinonyl, dihydroisoquinolinonyl, dihydrocoumarinyl, dihydrosiocoumarinyl, isoindolinonyl, benzodioxanyl, benzoxazolinonyl, pyrrolyl N-oxide, pyrimidinyl N-oxide, pyridazinyl N-oxide, pyridazinyl N-oxide, quniolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide. Exemplary heteroaryl groups include imidazolyl, pyrazolyl, thiadiazolyl, triazolyl, isoxazolyl, isothiazolyl, imidazolyl, thiazolyl, oxadiazolyl, and pyridyl. Other exemplary heteroaryl groups include 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-heteroaryl oxazolyl, 3-isoxazolyl, 4-isoxazolyl, 5-isoxazolyl, 2-thiazolyl, 4-thiazolyl, 5-thiazolyl, 2-furyl, 3-furyl 2-thienyl, 3-thienyl, 2-pyridyl, 3-pyridyl, pyridin-4-yl, 2-pyrimidyl, 4-pyrimidyl, 5-benzothiazolyl, purinyl, 2-benzimidazolyl, 5-indolyl, 1-isoquinolyl, 5- isoquinolyl, 2-quinoxalinyl, 5-quinoxalinyl, 3-quinolyl, and 6-quinolyl. Substituents for each of the above noted aryl and heteroaryl ring systems are selected from the group of acceptable aryl group substituents described below.

For brevity, the terms “aryl” when used in combination with other terms (e.g., aryloxy, arylthioxy, arylalkyl) includes both aryl and heteroaryl rings as defined above.

Each of the above terms (e.g., “alkyl”, “cycloalkyl”, “heteroalkyl”, heterocycloalkyl”, “aryl” and “heteroaryl”) are meant to include both substituted and unsubstituted forms of the indicated radical. The term “substituted” for each type of radical is explained below. When a compound of the present disclosure includes more than one substituent, then each of the substituents is independently chosen.

The term “substituted” in connection with alkyl, alkenyl, alkynyl, cycloalkyl, heteroalkyl and heterocycloalkyl radicals (including those groups referred to as alkylene, heteroalkylene, heteroalkenyl, cycloalkenyl, heterocycloalkenyl, and the like) refers to one or more substituents, wherein each substituent is independently selected from, but not limited to, 3- to 10-membered heteroalkyl, C₃-C₁₀ cycloalkyl, 3- to 10-membered heterocycloalkyl, aryl, heteroaryl, —OR^{a}, —SR^{a}, ═O, ═NR^{a}, ═N—OR^{a}, —NR^{a}R^{b}, -halogen, —SiR^{a}R^{b}R^{c}, —OC(O)R^{a}, —C(O)R^{c}, —C(O)OR^{a}, —C(O)NR^{a}R^{b}, —OC(O)NR^{a}R^{b}, NR^{c}C(O)R^{c}, —NR^{c}C(O)NR^{a}R^{b}, —NR^{c}C(S)NR^{a}R^{b}, —NR^{c}C(O)OR^{a}, —NR^{c}C(NR^{a}R^{b})═NR^{d}, —S(O)R^{c}, —S(O)₂R^{c}, —S(O)₂NR^{a}R^{b}, NR^{c}S(O)₂R^{a}, —CN and —NO₂, R^{a}, R^{b}, R^{c}, R^{d} and R² each indepenently refer to hydrogen, C₁-C₂₄ alkyl (e.g., C₁-C₁₀ alkyl or C₁-C₆ alkyl), C₃-C₁₀ cycloalkyl, C₁-C₂₄ heteroalkyl (e.g., C₁-C₁₀ heteroalkyl or C₁-C₆ heteroalkyl). C₃-C₁₀ heterocycloakyl, aryl, heteroaryl, arylalkyl and heteroarylalkyl, wherein, in one embodiment, R^{c} is not hydrogen. When two of the above R groups (e.g., R^{a} and R^{b}) are attached to the same nitrogen atom, they can be combined with the nitrogen atom to form a 5-, 6-, or 7-membered ring. For example, —NR^{a}R^{b} is meant to include pyrrolidinyl, N -alkyl-piperidinyl and morpholinyl.

The terms “substituted”in connection with aryl and heteroaryl groups, refers to one or more substituents, wherein each substituent is independently selected from, but not limited to, alkyl (e.g., C₁-C₂₄ alkyl, C₁-C₁₀ alkyl or C₁-C₆ alkyl), cycloalkyl (e.g., C₃-C₁₀ cycloalkyl, or C₃-C₈ cycloalkyl), alkenyl (e.g., C₁-C₁₀ alkenyl or C₁-C₆ alkenyl), alkynyl (e.g., C₁-C₁₀ alkynyl or C₁-C₆ alkynyl), heteroalkyl (e.g., 3- to 10-membered heteroalkyl), heterocycloalkyl (e.g., C₃-C₈ heterocycloalkyl), aryl, heteroaryl, —R^{a}, —OR^{a}, —SR^{a}, ═O, ═NR^{a}, ═N—OR^{a}, —NR^{a}R^{b}, -halogen, —SiR^{a}R^{b}R^{c}, —OC(O)R^{a}, —C(O)R^{c}, —C(O)OR^{a}, —C(O)NR^{a}R^{b}, —OC(O)NR^{a}R^{b}, —NR^{c}C(O)R^{a}, —NR^{c}C(O)NR^{a}R^{b}, —NR^{c}C(S)NR^{a}R^{b}, —NR^{c}C(O)OR^{a}, —NR^{c}C(NR^{a}R^{b})═NR^{d}, —S(O)R^{a}, —S(O)₂R^{c}, —S(O)₂NR^{a}R^{b}, —NR^{c}S(O)₂R^{a}, —CN, —NO₂-N₃, —CH(Ph)₂, fluoro(C₁-C₄)alkoxy, and fluoro(C₁-C₄)alkyl, in a number ranging from zero to the total number of open valences on the aromatic ring system, wherein R^{a}, R^{b}, R^{c}, R^{d} and R^{c} each independently refer to hydrogen, C₁-C₂₄ alkyl (e.g., C₁-C₁₀ alkyl or C₁-C₆ alkyl), C₃-C₁₀ cycloalkyl, C₁ -C₂₄ heteroalkyl (e.g., C₁-C₁₀ heteroalkyl or C₁-C₆ heteroalkyl), C₃-C₁₀ heterocycloalkyl, aryl, heteroaryl, arylalkyl and heteroarylalkyl, wherein, in one embodiment, R^{c} is not hydrogen. When two R groups (e.g., R^{a} and R^{b}) are attached to the same nitrogen atom, they can be combined with the nitrogen atom to form a 5-, 6-, or 7-membered ring. For example, —NR^{a}R^{b} is meant to include pyrrolidinyl, N-alkyl-piperidinyl and moropholinyl.

The terms “substituted” in connection with aryl and heteroaryl groups also refers to one or more fused ring(s), in which two hydrogen atoms on adjacent atoms of the aryl or heteroaryl ring are optionally replaced with a substituent of the formula —T-C(O)— (CRR′)_{q}—U—, wherein T and U are independently —NR—, —O—, —CRR′—or a single bond, and q is an integer from 0 to 3. Alternatively, two of the hydrogen atoms on adjacent atoms of the aryl or heteroaryl ring can optionally be replaced with a substituent of the formula —A-(CH₂)ᵣ—B—, wherein A and B are independently —CRR′—, —O—, —NR—, —S—, —(O)—, —S(O)₂), —S(O)₂NR′— or a single bond, and r is an integer from 1 to 4. One of the single bonds of the ring so formed can optionally be replaced with a double bond. Alternatively, two of the hydrogen atoms on adjacent atoms of the aryl or heteroaryl ring can optionally be replaced with a substituent of the formula —(CRR′)ₛ—X—(CR″R′″)_{d}—, where s and d are independently integers from 0 to 3, and X is —O—, —NR′—, —S—, —S(O)—, —S(O)₂—, or —S(O)₂NR′—, wherein the substituents R R′, R″ and R′″ in each of the formulas above are independently selected from hydrogen and (C₁-C₆)alkyl.

The terms “halo” or “halogen,” by themselves or as part of another substituent, mean at least one of fluorine, chlorine, bromine and iodine.

By “haloalkyl”is meant an alkyl radical, wherein alkyl is as defined above and wherein at least one hydrogen atom is replaced by a halogen atom. The term “haloalkyl,” is meant to include monohaloalkyl and polyhaloalkyl. For example, the terms “halo(C₁-C₄)alkyl” or “(C₁-C₄)haloalkyl” is mean to include, but not limited to, chloromethyl, 1-bromomethyl, fluoromethyl, difluoromethyl, trifluoromethyl, 1,1,1-trifluoromethyl and 4-chlorobutyl, 3-bromopropyl.

As used herein, the term “acyl” described the group —C(O)R^{c}, wherein R^{c} is selected from hydrogen, C₁-C₂₄ alkyl (e.g., C₁-C₁₀ alkyl or C₁-C₆ alkyl), C₁-C₂₄ alkenyl (e.g., C₁-C₁₀ alkenyl or C₁-C₆ alkenyl), C₁-C₂₄ alkynyl (e.g., C₁-C₁₀ alkynyl or C₁-C₆ alkynyl), C₃-C₁₀ cycloalkyl, C₁-C₂₄ heteroalkyl (e.g., C₁-C₁₀ heteroalkyl or C₁-C₆ heteroalkyl), C₃-C₁₀ heterocycloalkyl, aryl, heteroaryl, arylalkyl and heteroarylalkyl. In one embodiment, R^{c} is not hydrogen.

By “alkanoyl” is meant an acyl radical —C(O)—Alk-, wherein Alk is an alkyl radical as defined herein. Examples of alkanoyl include acetyl, propionyl, butyryl, isobutyryl, valeryl, isovaleryl, 2- methyl-butyryl, 2,2-dimethylpropinyl, hexanoyl, heptanol, octanoyl and the like.

As used herein, the term “heteroatom” includes oxygen (O), nitrogen (N), sulfur (S), silicon (Si) boron (B) and phosphorus (P). In one embodiment, heteroatoms are O, S and N.

By “oxo” is meant the group ═O.

By “sulfonyl” or “sulfonyl group” is meant a group that is connected to the remainder of a molecule via a —S(O)₂— moiety. Hence sulfonyl can be —S(O)₂R, wherein R is e.g., NHR′, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. An exemplary sulfonyl group is S(O)₂-Cy, wherein Cy is, e.g., substituted or unsubstituted aryl or substituted or unsubstituted heteroaryl.

By “sulfinyl” or “sulfinyl group” is meant a group that is connected to the remainder of the molecule via a —S(O)— moiety. Hence, sulfinyl can be —S(O)R, wherein R is as defined for sulfonyl group.

By “sulfonamide” is meant a group having the formula —S(O)₂NRR, where each of the R variables are independently selected from the variables listed above for R.

The symbol “R” is a general abbreviation that represents a substituent group as described herein. Exemplary substituent groups include alkyl, alkenyl, alkynyl, cycloalkyl, heteroalkyl, aryl, heteroaryl and heterocycloalkyl groups, each as defined herein.

As used herein, the term “aromatic ring” or “non-aromatic ring” is consistent with the definition commonly used in the art. For example, aromatic rings include phenyl and pyridyl. Non-aromatic rings include cyclohexanes.

As used herein, the term “fused ring system” means at least two rings, wherein each ring has at least 2 atoms in common with another ring. “Fused ring systems can include aromatic as well as non-aromatic rings. Examples of “fused ring systems” are naphthlenes, indoles, quinolines, chromenes and the like. Likewise, the term “fused ring” refers to a ring that has at least two atoms in common with the ring to which it is fused.

Where multiple substituents are indicated as being attached to a structure, those substituents are independently chosen. For example “ring A is optionally substituted with 1, 2 or 3 R_{q} groups” indicates that ring A is substituted with 1, 2 or 3 R_{q} groups, wherein the R_{q} groups are independently chosen (i.e., can be the same or different).

Where substituent groups are specified by their conventional chemical formulae, written from left to right, they equally encompass the chemically identical substituents, which would result from writing the structure from right to left. For example “—CH₂O—” is intended to also recite “—OCH₂—”.

### Catalysts

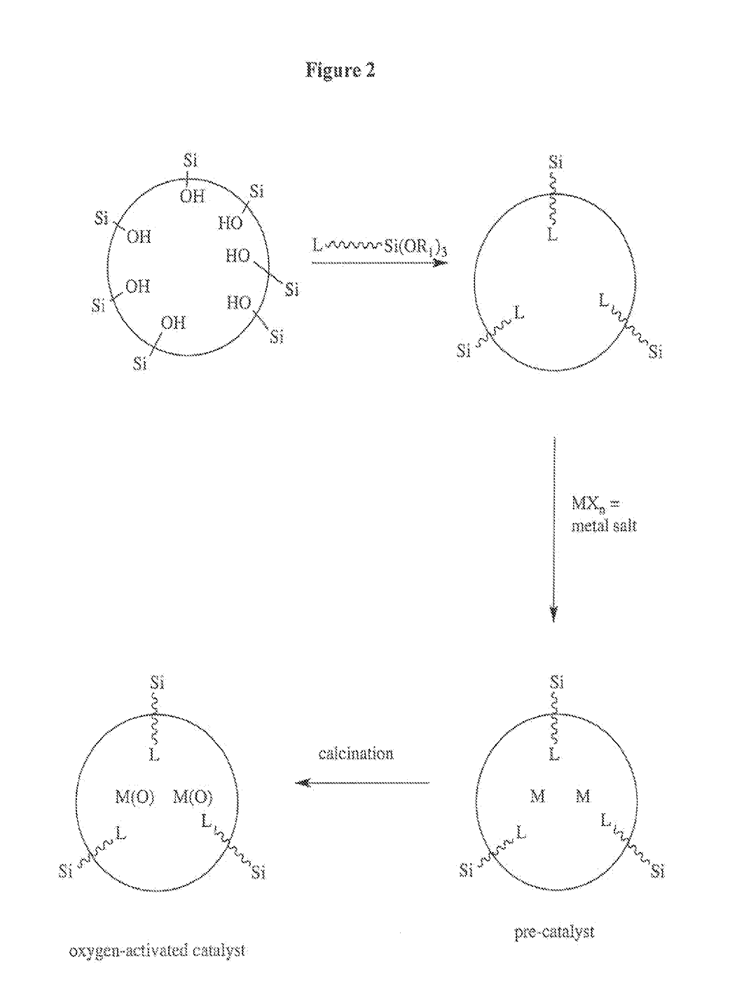

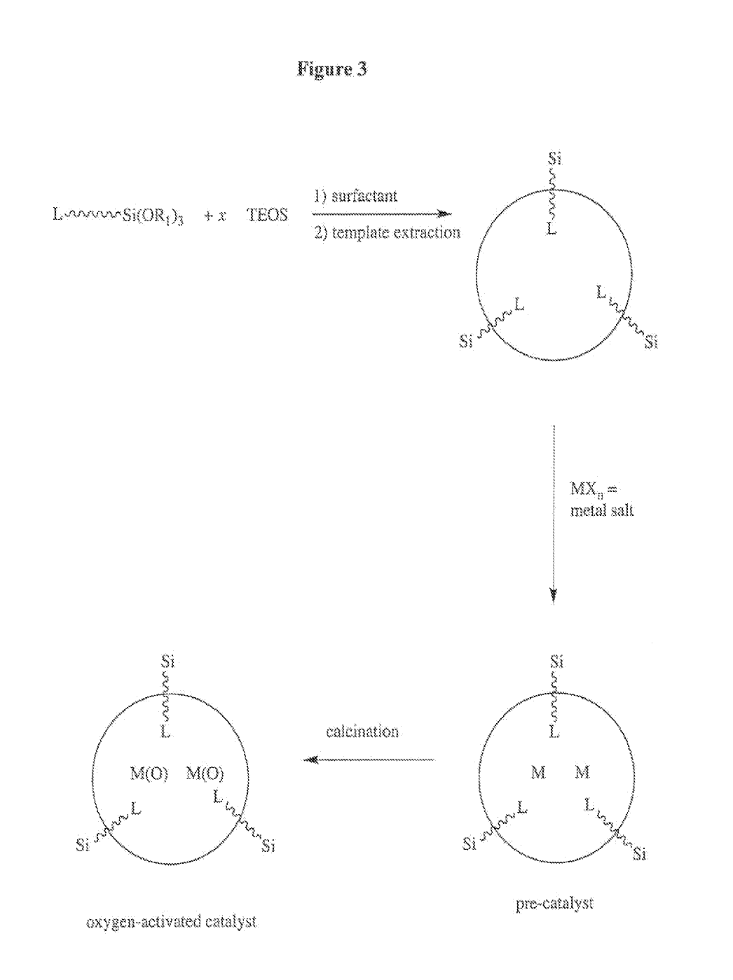

In certain embodiments, the catalyst synthesis takes place in several steps. FIG. 2 illustrates an exemplar post-synthetic grafting route and FIG. 3 illustrates an exemplary self-assembly route.

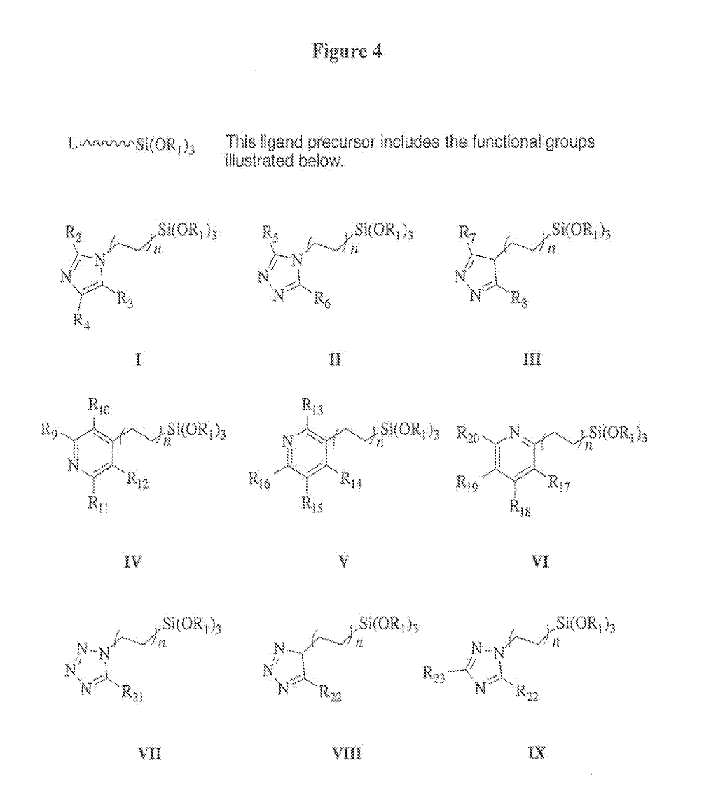

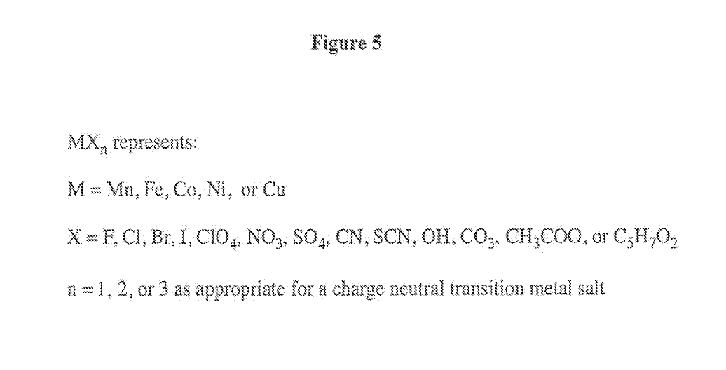

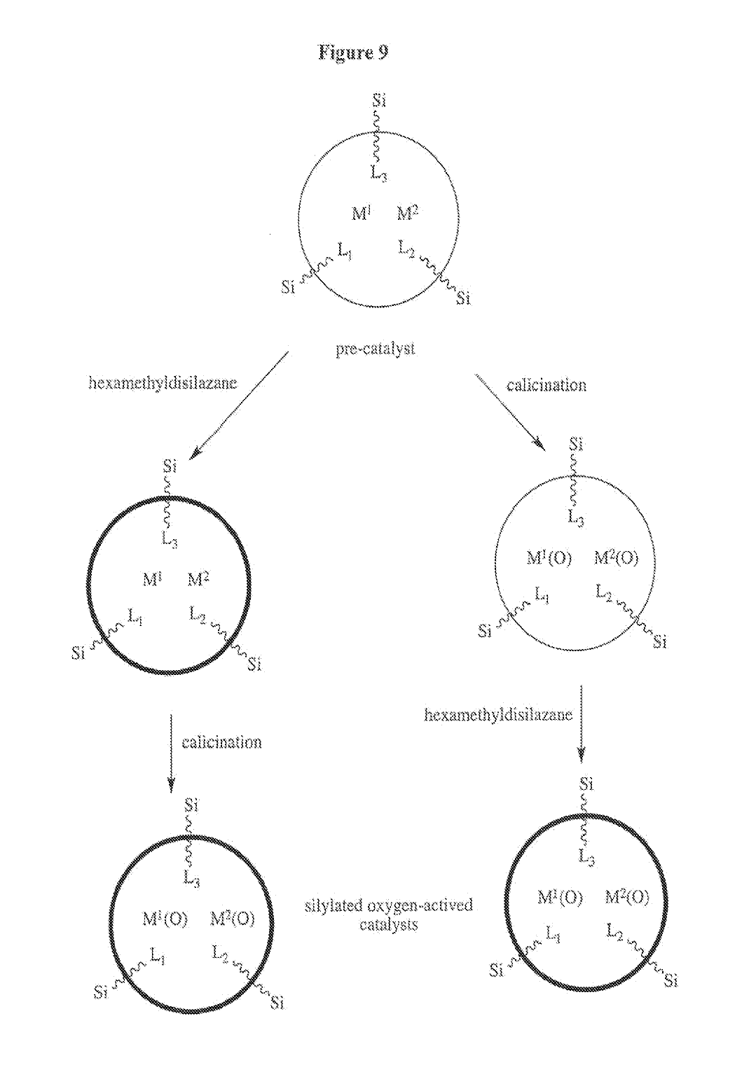

Post-synthetic grafted catalysts can be synthesized by first using a mesoporous silica template such as, but not limited to SBA-15 or MCM-41. The mesoporous silica is then reacted, e.g., as shown in FIG. 2, with an alkyl silyl ether containing a ligand precursor. Exemplary ligand precursors are shown in FIG. 4. This forms a ligand grafted mesoporous silica material that is then impregnated with a transition metal M, for example by coordination with a metal salt, MXₙ, forming the pre-catalyst, where M is, for example, Mn, Fe, Co, Ni, or Cu; X is F, Cl, Br, I, NO₃, CN, OH, CH₃COO, etc.; and n is, for example, 1-3. In certain illustrative embodiments, the metal salt can also have the formula, M_{y}Xₙ, where M is, for example, Mn, Fe, Co, Ni, or Cu; X is F, Cl, Br, I, NO₃, CN, OH, CH₃COO, etc.; and n is, for example, 1-3, and Y is 1-2 Exemplary metal salts are shown in FIG. 5. The pre-catalyst is heated in an oxidizing environment. In an exemplary method, a catalyst is pre-treated by heating the catalyst in a gaseous environment with continuous gas flow and at a pre-treatment temperature range of about 370 degrees Celsius to about 950 degrees Celsius. This forms the oxygen-activated catalyst. The oxygen-activated catalyst may then be silylated, for example, using methods outlined in FIG. 9 to form a silylated oxygen-activated catalyst.

In certain illustrative embodiments, self-assembled catalysts can be synthesized, for example, as illustrated in FIG. 3. In one embodiment, an alkyl silyl ether containing the ligand precursor is reacted with a stoichiometric amount of TEOS (tetraethyl orthosilicate) where x=4−24 and x is chosen to influence both the pore structure and size in the mesoporous silica material. A structure-direction agent, for example, an amine-based surfactant is added. Exemplary amine-based surfactants include n-alkyl amines, such as C₆-C₂₀ n-alkyl amines. In some illustrative embodiments, the amine-based surfactant is n-hexadecylamine and n-octadecylamine. Exemplary ligand precursors are shown in FIG. 4. This forms a ligand grafted mesoporous silica material that is then impregnated with metal M, for example, by coordination with a metal salt, for example, MXₙ, forming the pre-catalyst. Exemplary metal salts are shown in FIG. 5. The pre-catalyst is then heated in an oxidizing environment. In this method a catalyst is pre-treated by heating the catalyst in a gaseous environment with continuous gas flow and at a pre-treatment temperature range of about 370 degrees Celsius to about 950 degrees Celsius. This forms the oxygen-activated catalyst. The oxygen-activated catalyst may then be silylated, for example, using methods outlined in FIG. 9 to form a silylated oxygen-activated catalyst.

The catalysts of the invention comprise at least one ligand, for example, covalently linked to the silica matrix, at least one transition metal, and oxygen. In some embodiments, the ligand is capable of binding (e.g., complexing/coordinating) a transition metal. In some embodiments, the transition metal is bound (e.g., coordinated) to oxygen. The catalysts can include more than one ligand and/or more than one transition metal. In some embodiments, the ligand comprises a moiety selected from an imidazole moiety, a triazole moiety (e.g., a 1,2,3-triazole moiety, or a 1,2,4-triazole moiety), a pyrazole moiety, a pyridine moiety (e.g., a 2-pyridine, 3-pyridine, or 4-pyridine moiety), an a tetrazole moiety.

### Ligand Precursors

One or more ligand precursor can be used to form the catalyst. In some embodiments, the ligand precursor comprises a moiety selected from an imidazole moiety, a triazole moiety (e.g., a 1,2,3-triazole moiety, or a 1,2,4-triazole moiety), a pyrazole moiety, a pyridine moiety (e.g., a 2-pyridine, 3-pyridine, or 4-pyridine moiety), and a tetrazole moiety.

In FIG. 4, exemplary ligand precursors having Formulae I-IX are illustrated. In some embodiments in formulae I-IX, R₁ is selected from C₁-C₆ alkyl. In other embodiments, R₁ is methyl or ethyl. In some embodiments, in Formulae I-IX, n=0-6. In other embodiments in Formulae I-IX, R₁ is selected from methyl and ethyl and n is 0-6.

In some embodiments, the ligand precursor comprises an imidazole moiety and has a structure according to Formula I, wherein R₂, R₃, and R₄ are independently selected from the group consisting of H, amino (e.g., alkyl amino), alkyl, substituted alkyl, heteroalkyl, substituted heteroalkyl, cycloalkyl, substituted cycloalkyl, heterocycloalkyl, substituted heterocyloalkyl, aryl, substituted aryl, heteroaryl, substituted heteroaryl, aralkyl, substituted aralkyl, hydroxyl, alkoxy, alkenyl, substituted alkenyl, alkynyl, substitute alkynyl, amide, azo, benzyl, substituted benzyl, carbonate, acyl, carboxylate, amide, sulfonamide, cyanate, ether, ester, halide, imine, isocyanide, isocyanate, ketone (oxy), sulfonyl, nitrile, nitro, nitroso, thiol, and substituted thiol (e.g., alkyl thiol).

In other embodiments, the ligand precursor includes a substituted 1,2,4-triazoles (4-N) moiety and has a structure according to Formula II, wherein R₅ and R₆ are independently selected from the group consisting of H, amino (e.g., alkyl amino), alkyl, substituted alkyl, heteroalkyl, substituted heteroalkyl, cycloalkyl, substituted cycloalkyl, heterocycloalkyl, substituted heterocyloalkyl, aryl, substituted aryl, heteroaryl, substituted heteroaryl, aralkyl, substituted aralkyl, hydroxyl, alkoxy, alkenyl, substituted alkenyl, alkynyl, substitute alkynyl, amide, azo, benzyl, substituted benzyl, carbonate, acyl, carboxylate, amide, sulfonamide, cyanate, ether, ester, halide, imine, isocyanide, isocyanate, ketone (oxy), sulfonyl, nitrile, nitro, nitroso, thiol, and substituted thiol (e.g., alkyl thiol).

In other embodiments, the ligand precursor includes a substituted pyrazole moiety and has a structure according to Formula III, wherein R₇ and R₈ are independently selected from the group consisting of H. amino (e.g., alkyl amino), alkyl, substituted alkyl, heteroalkyl, substituted heteroalkyl, cycloalkyl, substituted cycloalkyl, heterocycloalkyl, substituted heterocyloalkyl, aryl, substituted aryl, heteroaryl, substituted heteroaryl, aralkyl, substituted aralkyl, hydroxyl, alkoxy, alkenyl, substituted alkenyl, alkynyl, substitute alkynyl, amide, azo, benzyl, substituted benzyl, carbonate, acyl, carboxylate, amide, sulfonamide, cyanate, ether, ester, halide, imine, isocyanide, isocyanate, ketone (oxy), sulfonyl, nitrile, nitro, nitroso, thiol, and substituted thiol (e.g., alkyl thiol).

In other embodiments, the ligand precursor includes a substituted 4-pyridine moiety and has a structure according to Formula IV, wherein R₉, R₁₀, R₁₁, and R₁₂ are independently selected from the group consisting of H, amino (e.g., alkyl amino), alkyl, substituted alkyl, heteroalkyl, substituted heteroalkyl, cycloalkyl, substituted cycloalkyl, heterocycloalkyl, substituted heterocycloalkyl, aryl, substituted aryl, heteroaryl, substituted heteroaryl, aralkyl, substituted aralkyl, hydroxyl, alkoxy, alkenyl, substituted alkenyl, alkynyl, substitute alkynyl, amide, azo, benzyl, substituted benzyl, carbonate, acyl, carboxylate, amide, sulfonamide, cyanate, ether, ester, halide, imine, isocyanide, isocyanate, ketone (oxy), sulfonyl, nitrile, nitro, nitroso, thiol, and substitute thiol (e.g., alkyl thiol).

In other embodiments, the ligand precursor includes a substituted 3-pyridine moiety and has a structure according to Formula V, wherein R₁₃, R₁₄, R₁₅, and R₁₆ are independently selected from the group consisting of H, amino (e.g., alkyl amino), alkyl, substituted alkyl, heteroalkyl, substituted heteroalkyl, cycloalkyl, substituted cycloalkyl, heterocycloalkyl, substituted heterocyloalkyl, aryl, substituted aryl, heteroaryl, substituted heteroaryl, aralkyl, substituted aralkyl, hydroxyl, alkoxy, alkenyl, substituted alkenyl, alkynyl, substitute alkynyl, amide, azo, benzyl, substituted benzyl, carbonate, acyl, carboxylate, amide, sulfonamide, cyanate, ether, ester, halide, imine, isocyanide, isocyanate, ketone (oxy), sulfonyl, nitrile, nitro, nitroso, thiol, and substituted thiol (e.g., alkyl thiol).

In other embodiments, the ligand precursor includes a substituted 2-pyridine moiety and has a structure according to Formula VI, wherein R₁₇, R₁₈, R₁₉, and R₂₀ are independently selected from the group consisting of H, amino (e.g., alkyl amino), alkyl, substituted alkyl, heteroalkyl, substituted heteroalkyl, cycloalklyl, substituted cycloalkyl, heterocycloalkyl, substituted heterocycloalkyl, aryl, substituted aryl, heteroaryl, substituted heteroaryl, aralkyl, substituted aralkyl, hydroxyl, alkoxy, alkenyl, substituted alkenyl, alkynyl, substitute alkynyl, amide, azo, benzyl, substituted benzyl, carbonate, acyl, carboxylate, amide, sulfonamide, cyanate, ether, ester, halide, imine, isocyanide, isocyanate, ketone (oxy), sulfonyl, nitrile, nitro, nitroso, thiol, and substituted thiol (e.g., alkyl thiol).

In other embodiments, the ligand precursor includes a substituted tetrazole moiety and has a structure according to Formula VII, wherein R₂₁ is independently selected from the group consisting of H, amino (e.g., alkyl amino), alkyl, substituted alkyl, heteroalkyl, substituted heteroalkyl, cycloalkyl, substituted cycloalkyl, heterocycloalkyl, substituted heterocyloalkyl, aryl, substituted aryl, heteroaryl, substituted heteroaryl, aralkyl, substituted aralkyl, hydroxyl, alkoxy, alkenyl, substituted alkenyl, alkynyl, substitute alkynyl, amide, azo, benzyl, substituted benzyl, carbonate, acyl, carboxylate, amide, sulfonamide, cyanate, ether, ester, halide, imine, isocyanide, isocyanate, ketone (oxy), sulfonyl, nitrile, nitro, nitroso, thiol, and substituted thiol (e.g., alkyl thiol).

In other embodiments, the ligand precursor includes a substituted 1,2,3-triazole moiety and has a structure according to Formula VIII, wherein R₂₂ is independently selected from the group consisting of H, amino (e.g., alkyl amino), alkyl, substituted alkyl, heteroalkyl, substituted heteroalkyl, cycloalkyl, substituted cycloalkyl, heterocycloalkyl, substituted heterocyloalkyl, aryl, substituted aryl, heteroaryl, substituted heteroaryl, aralkyl, substituted aralkyl, hydroxyl, alkoxy, alkenyl, substituted alkenyl, alkynyl, substitute alkynyl, amide, azo, benzyl, substituted benzyl, carbonate, acyl, carboxylate, amide, sulfonamide, cyanate, ether, ester, halide, imine, isocyanide, isocyanate, ketone (oxy), sulfonyl, nitrile, nitro, nitroso, thiol, and substituted thiol (e.g., alkyl thiol).

In other embodiments, the ligand precursor includes a substituted 1,2,4-triazole (1-N) moiety and has a structure according to Formula IX, wherein R₂₂ and R₂₃ are independently selected from the group consisting of H, amino (e.g., alkyl amino), alkyl substituted alkyl, heteroalkyl substituted heteroalkyl, cycloalkyl, substituted, cycloalkyl, heterocycloalkyl, substituted heterocyloalkyl, aryl, substituted aryl, heteroaryl, substituted heteroaryl aralkyl, substituted aralkyl hydroxyl, alkoxy, alkenyl, substituted alkenyl, alkynyl, substitute alkynyl, amide, azo, benzyl substituted benzyl, carbonate, acyl, carboxylate, amide, sulfonamide, cyanate, ether, ester, halide, imine, isocyanide, isocyanate, ketone (oxy), sulfonyl, nitrite, nitro, nitroso, thiol, and substituted thiol (e.g., alkyl thiol).

### Transition Metals

The catalysts of the invention include at least one transition metal. In FIG. 5, exemplary transition metal salts that can be used to synthesize the pre-catalyst are presented. Exemplary transition metals include, but are not limited to, manganese, iron, cobalt, nickel, copper, and combinations thereof. In certain embodiments, metal salts are used in the formation of the pre-catalyst and may include a counteranion that may influence the eventual oxygen-activated catalyst structure and activity. Exemplary counteranions for the transition metal salts include, but are not limited to, fluoride, chloride, bromide, iodide, perchlorate, nitrate, sulfate, cyanide, thiocyanate, hydroxide, carboxylate, acetate, or acetylacetonate. Where appropriate, the transition metal salts used to synthesize the pre-catalysts may also contain waters of hydration.

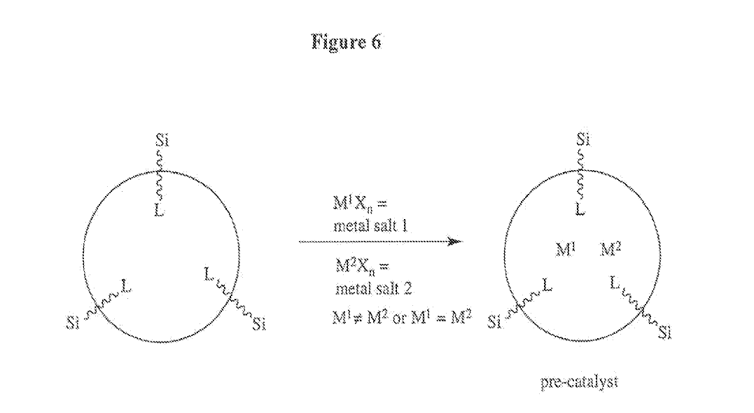

In certain embodiments, the catalysts of the invention can contain more than one transition metal. FIG. 6 illustrates an alternative exemplary configuration of the post-synthetic grafted pre-catalyst illustrated in FIG. 2. In this example, more than one metal salt may be used yielding bi-metallic catalytic species.

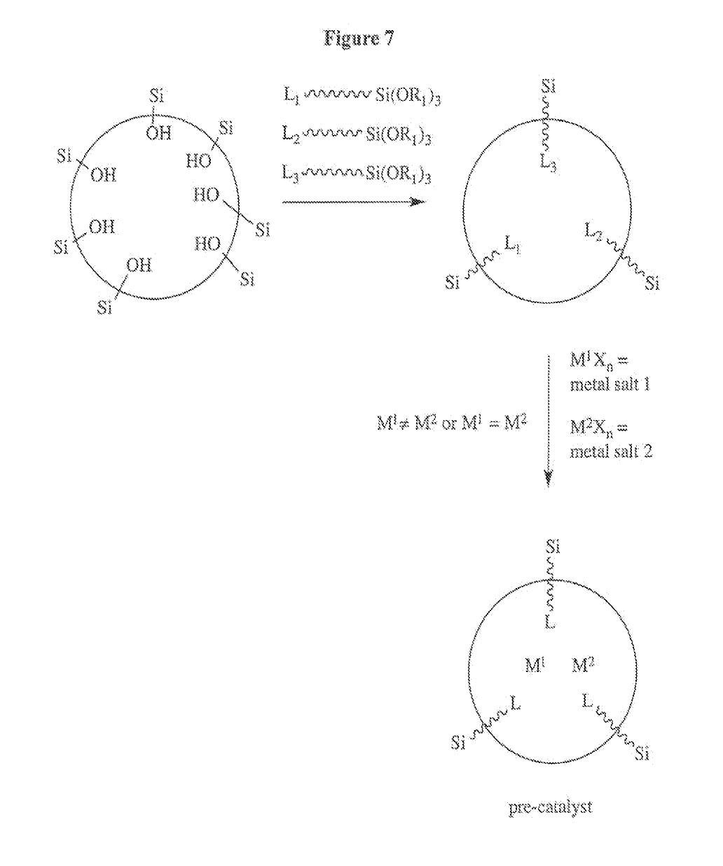

A further illustrative example of the post-synthetic grafted pre-catalyst is shown in FIG. 7. In this example, more than one ligand precursor is used to synthesize the post-synthetic grafted pre-catalysts. Therefore, mono-functional, bi-functional, and tri-functional post-synthetic grafted catalysts are possible.

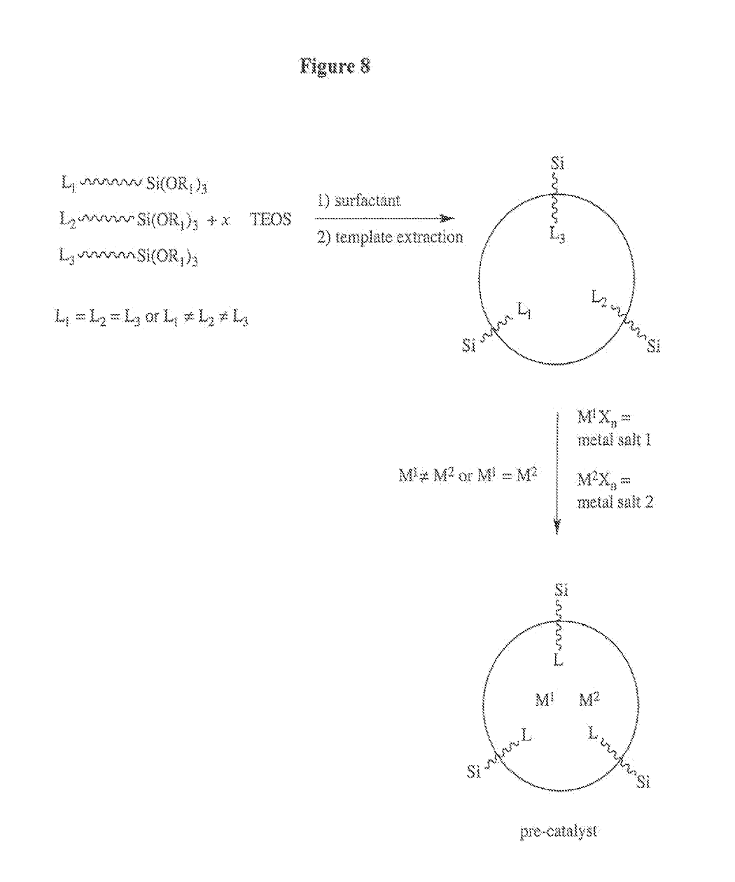

FIG. 8 illustrates an exemplary synthetic route to another example of self-assembled catalysts. In this example more than one ligand precursor is used to synthesize the self-assembled pre-catalysts. Therefore, mono-functional, bi-functional, and tri-functional self-assembled catalysts are possible. This example also illustrates that more than one metal salt may be used to synthesize the self-assembled pre-catalysts.

FIG. 9 illustrates two exemplary synthetic routes to silylate the surface of the oxygen-activated catalysts. Reagents used to silylate surfaces include, but are not limited to, hexamethyldisilazane. In one example, the hexamethyldisilazane reacts with the pre-catalyst prior to calcination. In another example, the silylation step occurs after calcination. Silylation of the surface may affect and enhance the oxygen-activated catalyst activity and selectivity. This class of catalysts is referred to as silylated oxygen-activated catalysts.

### EXAMPLES

### Example 1

### Preparation of Catalysts by Post-Synthetic Grafting

### A. Synthesis of Ligand Precursors

Ligand precursors can be synthesized using art recognized procedures, or using the procedures outlined below. It will be within the capabilities of a person of ordinary skill in the art to adapt the below procedures to prepare additional ligands, for example, those exemplary embodiments illustrated in FIG. 4.

(a) Synthesis of N-(3-propyltrimethoxysilane) imidazole (Ligand Precursor A)

To a solution of imidazole in dry toluene, 3-chloropropyltriethoxysilane was added and the mixture was refluxed overnight under a nitrogen atmosphere. The solvent was removed by rotatory evaporation under reduced pressure, and the product N-(3-propyltrimethoxysliane) imidazole was obtained as a transparent liquid after neutral column chromatography, eluting with hexane and ethyl ether (5:1). ¹H NMR(400 MHz, CDCl₃): δ 7.53 (s, 1H), 7.07 (s, 1H), 6.93 (s, 1H), 3.96 (t, J=7.5 Hz, 2H), 3.82 (q, J=7.0 Hz, 6H), 1.90 (m, 2H), 1.23 (t, J=7.0 Hz, 9H), 0.57 (t, J=8.0 Hz, 2H); ¹³C NMR (125 MHz, CDCl₃): δ 7.2, 18.1, 24.8, 48.9, 58.2, 118.6, 129.0, 137.0.

(b) Synthesis of N-(3-propyltrimethoxysilane)-1,2,4triazole (Ligand Precursor B)

SOCl₂ was added with stirring to DMF below ambient temperature. After stirring, to the solution of this mixture, was added slowly aqueous hydrazine hydrate in DMF. The mixture was stirred at ambient temperature for two days and a white precipitate of dimethylformamide azine dihydrochloride was collected by filtration and washed with DMF and Et₂O.

To a solution of (3-triethoxysilyl)-propan-1-amine) in benzene was added the above dimethylformanhde-axine-dihydrochloride and TsOH and the mixture was heated. The product precipitated from solution. The supernatant was tritirated with diethyl ether affording further precipitate. The solids were collected and washed with hexanes and dried under vacuum to yield a waxy off white solid.

### B. Preparation of Silica Matrix/Templates/Substrates

Silica substrates can be synthesized using art recognized methods, or using the procedures outlined below. It will be within the capabilities of a person of ordinary skill in the art to adapt the below procedures to prepare additional substrates.

### (a) Preparation of SBA-15

P123 (commercially available) was dissolved in an aqueous solution of HCl. The resulting clear solution was then added to TEOS. The mixture was stirred at room temperature until a transparent solution appeared. After gently heating the solution, NaF was added. After stirring above ambient temperature for several days, the resulting powder was filtered off and the surfactant was removed by Soxhlet extraction over ethanol for 24 hours. After drying with heating under vacuum, SBA-15 was obtained.

### C. Post-Synthetic Grafting of Silica Templates

To a suspension of SBA-15 in a suitable solvent (e.g., toluene) one or more ligand precursor was added. The mixture was typically refluxed and stirred (e.g., for about 24 hours). After filtration, the solid was washed with a suitable solvent (e.g., acetone and/or diethyl ether) and dried (e.g., at 120° C.) under vacuum to give a ligand-grafted silica template.

### (a) Post-Synthetic Grafting of SBA With Ligand Precursors A and B

To a suspension of SBA in toluene, ligand precursor A and ligand precursor B were added. The mixture was refluxed and stirred. After filtration, the solid was washed and then dried with heating under vacuum to give a white powder.

### (b) Post-Synthetic Grafting of SBA With Ligand Precursor A

To a suspension of SBA in toluene, ligand precursor A was added. The mixture was refluxed and stirred. After nitration, the solid was washed and then dried with heating under vacuum to give a white powder.

### (b) Post-Synthetic Grafting of SBA With Ligand Precursor B

To a suspension of SBA in toluene, ligand precursor B was added. The mixture was refluxed and stirred. After filtration, the solid was washed and then dried with heating under vacuum to give a white powder.

### D. Metal Impregnation

Grafted mesoporous silica and a transition metal salt (i.e., MXₙ) were combined in THF and heated to reflux. The solid was collected by filtration, washed with THF and water, and dried with heating under vacuum overnight.

### E. Preparation of Oxygen-Activated Catalysts (Calcination)

The materials were calcinated at 700° C. for several hours under oxygen atmosphere in a tube furnace (Thermo Scientific).

### F. Methane to Methanol Conversion and Testing

Catalytic reactions were carried out using a high pressure reactor. Catalyst was added to a borosilicate glass vial. A mixture of methane and oxygen in a ratio of 1:1 under a total pressure of 2-12 atm was passed through the high pressure reactor. The reactor was heated to 260° C. for 1-24 hours.

To rigorously demonstrate that the systems produced methanol and were in fact catalytic, detailed spectroscopic experiments were conducted including ¹H NMR of the reaction products as well as calibration of the product distribution, mass balance, and methane and oxygen consumption by GC-MS that was internally calibrated using internal standards and constructing calibration curves. ¹H NMR alone can be insufficient to make this determination as paramagnetic impurities may be present which would cause a shift in the observed resonance frequencies.

For NMR-analysis, after cooling down the reaction, the vial was rinsed with D₂O and the solution was analyzed by ¹H NMR. For GC analysis, the reactor was coupled to a GC and the gas phase mixture was analyzed, and the retention times were compared to runs with pure standards. The yields and selectivity were calculated by integrating the GC peak areas and quantifying them against calibration curves constructed from pure standards.

Using the above procedures, the following exemplary catalysts were prepared and tested, and were found to be active:

1. Post-synthetic grafted triazole silica impregnated with copper

2. Post-synthetic grafted triazole silica impregnated with manganese

3. Post-synthetic grafted triazole silica impregnated with copper and manganese

4. Post-synthetic grafted imidazole silica impregnated with copper

5. Post-synthetic grafted imidazole silica impregnated with manganese

6. Post-synthetic grafted imidazole silica impregnated with copper and manganese

7. Post-synthetic grafted imidazole-triazole silica impregnated with copper

8. Post-synthetic grafted imidazole-triazole silica impregnated with manganese

9. Post-synthetic grafted imidazole-triazole silica impregnated with copper and manganese

The following additional exemplary catalysts can be prepared using the above procedures:

1. Post-synthetic grafted tetrazole silica impregnated with copper

2. Post-synthetic grafted tetrazole silica impregnated with manganese

3. Post-synthetic grafted tetrazole silica impregnated with copper and manganese

4. Post-synthetic grafted pyrazole silica impregnated with copper

5. Post-synthetic grafted pyrazole silica impregnated with manganese

6. Post-synthetic grafted pyrazole silica impregnated with copper and manganese

7. Post-synthetic grafted pyridine silica impregnated with copper

8. Post-synthetic grafted pyridine silica impregnated with manganese

9. Post-synthetic grafted pyridine silica impregnated with copper and manganese

Additional catalysts may be prepared by incorporating a transition metal other than copper or manganese (e.g., iron, cobalt, or nickel) into each of the above catalysts, e.g., instead of or in addition to copper or manganese.

### Example 2

### Preparation of Self-Assembled Silica Catalysis

### A. Preparation of Self-Assembled Silica

In a typical preparation, a mixture of silylated ligand and tetraethyl orthosilate (TEOS) was added under stirring to a solution of n-hexadecylamine in a 55:45 EtOH (95%)-H₂O mixture at 35° C. A white precipitate appears within some minutes. The reaction mixture was kept at slightly above ambient temperature for several hours. The solid was then filtered and n-hexadecylamine was removed by Soxhlet extraction. After drying with heating under vacuum, the self-assembly mesoporous silica material was isolated.

### D. Metal Impregnation, Calcination and Testing

Self-assembled silica and a transition metal salt (e.g., MXₙ) were combined in THF and heated to reflux for several hours. The solid was collected by filtration and washed, and dried with heating under vacuum overnight. Calcination and testing was performed as outlined in Example 1.

The following catalysts were synthesized using the above procedures and were found to be active:

1. Self-assembled imidazole silica impregnated with copper

2. Self-assembled imidazole silica impregnated with manganese

3. Self-assembled imidazole silica impregnated with copper and manganese

The following additional catalysts can be prepared using the above procedures:

1. Self-assembled tetrazole silica impregnated with copper

2. Self-assembled tetrazole silica impregnated with manganese

3. Self-assembled tetrazole silica impregnated with copper and manganese

4. Self-assembled pyrazole silica impregnated with copper

5. Self-assembled pyrazole silica impregnated with manganese

6. Self-assembled pyrazole silica impregnated with copper and manganese

7. Self-assembled pyridine silica impregnated with copper

8. Self-assembled pyridine silica impregnated with manganese

9. Self-assembled pyridine silica impregnated with copper and manganese

10. Self-assembled triazole silica impregnated with copper

11. Self-assembled triazole silica impregnated with manganese

12. Self-assembled triazole silica impregnated with copper and manganese

Additional catalysts may be prepared by incorporating a transition metal other than copper or manganese (e.g., iron, cobalt or nickel) into each of the above catalysts, e.g., instead of or in addition to copper or manganese.

As one of ordinary skill in the art will appreciate, various changes, substitutions and alterations could be made or otherwise implemented without departing from the principles of the invention. Accordingly, the scope of the invention should be determined by the following claims and their legal equivalents.

What is claimed is:

1. A catalyst comprising:
i. a solid matrix;
ii. at least one transition metal;
iii. at least one ligand eovalently bound to the solid matrix; and
iv. oxygen bound to the transition metal.

2. The catalyst of claim 1, wherein said ligand is bound to said transition metal.

3. The catalyst of claim 1 or 2, wherein said solid matrix is a silica matrix.

4. The catalyst of claim 3, wherein said silica matrix is mesoporous or nanoporous silica.

5. The catalyst of any one of claims 1 to 4, wherein said transition metal is selected from the group consisting of manganese, iron, cobalt, nickel, copper, and combinations thereof.

6. The catalyst of any one of claims 1 to 5, wherein said ligand comprises a moiety selected from an imidazole moiety, a triazole moiety, a pyrazole moiety, a pyridine moiety, and a tetrazole moiety.

7. The catalyst of claim 6, wherein said imidazole moiety, said triazole moiety, said pyrazole moiety, said pyridine moiety, and said tetrazole moiety are selected form those depicted within FIG. 4.

8. A method for synthesizing an oxygen-activated catalyst, the method comprising: (i) contacting a pre-catalyst with oxygen (calcination) in a gaseous environment, thereby forming said oxygen-activated catalyst, wherein the pre-catalyst comprises (a) a solid matrix; (b) at least one transition metal; and (c) at least one ligand covalently bound to said solid matrix.

9. The method of claim 8, wherein said ligand is bound to said transition metal.

10. The method of claim 8 or 9, wherein said contacting said pre-catalyst with said oxygen occurs at a temperature from about 370° C. to about 950° C.

11. The method of any one of claims 8 to 10, wherein said solid matrix is a silica matrix.

12. The method of claim 11, wherein said silica matrix is mesoporous or nanoporous silica.

13. The method of any one of claims 8 to 12 further comprising: (ii) reacting said solid matrix with a ligand precursor, thereby forming a ligand-grafted solid matrix.

14. The method of claim 13, wherein said solid matrix is a mesoporous silica template selected from SBA-15 and MCM-41.

15. The method of claim 13 or 14, wherein said ligand precursor comprises an imidazole moiety, a triazole moiety, a pyrazole moiety, a pyridine moiety, or a tetrazole moiety.

16. The method of claim 15, wherein said ligand precursor further comprises a silyl ether moiety.

17. The method of claim 16, wherein said ligand precursor has a structure according to Formula I, Formula II, Formula III, Formula IV, Formula V, Formula VI, Formula VII, Formula VIII, or Formula IX as shown in FIG. 4, wherein R₁ to R₂₃ are independently selected from H, amino, alkyl, substituted alkyl. heteroalkyl, substituted heteroalkyl, cycloalkyl, substituted cycloalkyl heterocycloalkyl, substituted heterocyloalkyl, aryl, substituted aryl, heteroaryl, substitute heteroaryl, aralkyl, substituted aralkyl, hydroxyl, alkoxy, alkenyl, substituted alkenyl, alkynyl, substitute alkynyl, amide, azo, benzyl, substituted benzyl, carbonate, acyl, carboxylate, amide, sulfonamide, cyanate, ether, ester, halide, imine, isocyanide, isocyanate, oxy, sulfonyl, nitrile, nitro, nitroso, thiol, and substituted thiol.

18. The method of claim 17, wherein said ligand precursor is selected from N-(3-propyltrimethoxysilane) imidazole and N-(3-propyltrimethoxysilane) 1,2,4,-triazole.

19. the method of any one of claims 13 to 18 further comprising: (iii) reacting said ligand-grafted solid matrix with a transition metal salt, thereby forming said pre-catalyst.

20. the method of claim 19, wherein said transition metal is selected from the group consisting of manganese, iron, cobalt, nickel, copper, and combinations thereof.

22. The method of claim 8 further comprising (ii) reacting a ligand precursor with tetraethyl orthosilate (TEOS) at a ratio of TEOS:ligand precursor from about 4 to 24; and optionally adding a structure-directing agent, thereby forming a ligand-grafted silica matrix.

23. The method of claim 22, wherein said structure-directing agent is an amine-based surfactant.

24. The method of claim 23, wherein said amine-based surfactant is selected from n-hexadecylamine and n-octadecylamine.

25. The method of any one of claims 22 to 24, wherein said ligand precursor comprises an imidazole moiety, a triazole moiety, a pyrazole moiety, a pyridine moiety, or a tetrazole moiety.

26. The method of claim 25, wherein said ligand precursor further comprises a silyl ether moiety.

27. The method of claim 26, wherein said ligand precursor has a structure according to Formula I, Formula II, Formula III, Formula IV, Formula V, Formula VI, Formula VII, Formula VIII, or Formula IX as shown in FIG. 4, wherein R₁ to R₂₃ are independently selected from H, amino, alkyl, substituted alkyl, heteroalkyl, substituted heteroalkyl, cycloalkyl, substituted cycloalkyl, heterocycloalkyl, substituted heterocycloalkyl, aryl, substituted aryl, heteroaryl, substituted heteroaryl, aralkyl, substituted aralkyl, hydroxyl, alkoxy, alkenyl, substituted alkenyl, alkynyl, substitute alkynyl, amide, azo, benzyl, substituted benzyl, carbonate, acyl, carboxylate, amide, sulfonamide, cyanate, ether, ester, halide, imine, isocyanide, isocyanate, oxy, sulfonyl, nitrile, nitro, nitroso, thiol, and substituted thiol.

28. The method of claim 27, wherein said ligand precursor is selected from N-(3-propytrimethoxysilane) imidazole and N-(3-propyltrimethoxysilane) 1,2,4-triazole.

29. The method of any one of claims 22-28 further comprising, reacting said ligand-grafted silica matrix with a transition metal salt, thereby forming said pre-catalyst.

30. The method of claim 29, wherein said transition metal is selected from the group consisting of manganese, iron, cobalt, nickel, copper, and combinations thereof.

31. The method of claim 30, wherein said transition metal is selected from the group consisting of manganese, copper, and combinations thereof.

32. The method of any one of claims 8 to 31, further comprising silylating said pre-catalyst or said oxygen-activated catalyst thereby forming a silylated pre-catalyst or a silylated oxygen-activated catalyst.

33. An oxygen-activated catalyst made according to the method of any one of claims 8 to 32.

34. A method for directly converting methane (CH₄) to methanol (CH₃—OH) comprising, contacting a gas feed comprising methane with an oxygen-activated catalyst according to any one of claims 1 to 7 and 33, under conditions sufficient to form said methanol.

35. The method of claim 34, wherein said gas feed is contacted with said oxygen-activated catalyst at a temperature below about 750° C.

36. The method of claim 35, wherein said gas feed is contacted with said oxygen-activated catalyst at a temperature from about 150° C. to about 350° C.

37. The method of any one of claims 34 to 36, wherein said gas feed is contacted with said oxygen-activated catalyst at a pressure of less than about 50 atm.

38. The method of claim 37, wherein said gas feed is contacted with said oxygen-activated catalyst at a pressure of less than about 20 atm.

39. The method of claim 38, wherein said gas feed is contacted with said oxygen-activated catalyst at ambient (atmospheric) pressure.

40. The method of any one of claim 34 to 39 wherein said gas feed further comprises oxygen.

41. the method of any one of claim 34 to 40 wherein said gas feed further comprises a carrier gas.

42. The method of any one of claim 34 to 41 further comprising, collecting said methanol.

43. A method for directly converting methane to methanol at a temperature of less than 750° C., said method comprising: contacting a gas feed comprising methane with an oxygen-activated catalyst, thereby forming said methanol from said methane, wherein said oxygen-activated catalyst comprises:
i. a solid matrix;
ii. at least one transition metal;
iii. at least one ligand covalently bound to said solid matrix; and
iv. oxygen bound to said transition metal.

44. The method of claim 43, wherein said ligand is bound to said transition metal.

45. The method of claim 43 or 44, wherein said solid matrix is a silica matrix.

46. The method of claim 45, wherein said silica matrix is mesoporous or nanoporous silica.

47. The method of any one of claims 43 to 46, wherein said transition metal is selected from the group consisting of manganese, iron, cobalt, nickel, copper, and combinations thereof.

48. the method of claim 47, wherein said transition metal is selected from the group consisting of manganese, copper, and combinations thereof.

49. The method of any one of claims 43 to 48, wherein the ligand comprises a moiety selected from an imidazole moiety, a triazole moiety, a pyrazole moiety, a pyridine moiety, and a tetrazole moiety.

50. The method of claim 49, wherein said imidazole moiety, said triazole moiety, said pyrazole moiety, said pyridine moiety, and said tetrazole moiety are selected from those depicted in FIG. 4, wherein R₁ and R₂₃ are independently selected from H, amino, alkyl, substituted alkyl, heteroalkyl, substituted heteroalkyl, cycloalkyl, substituted cycloalkyl, heterocycloalkyl, substituted heteroalkyl, cycloalkyl, substituted aryl, heteroaryl, substituted heteroaryl, aralkyl, substituted aralkyl, hydroxyl, alkoxy, alkenyl, substituted alkenyl, alkynyl, substitute alkynyl, amide, azo, benzyl, substituted benzyl, carbonate, acyl, carboxylate, amide, sulfonamide, cyanate, ether, ester, halide, imine, isocyanide, isocyanate, oxy, sulfonyl, nitrile, nitro, nitroso, thiol, and substituted thiol.

51. The method of any one of claims 43 to 50, wherein said gas feed is contacted with said oxygen-activated catalyst at a pressure of less than about 50 atm.

52. The method of claim 51, wherein said gas feed is contacted with said oxygen-activated catalyst at a pressure of less than about 20 atm.

53. The method of claim 52, wherein said pressure is ambient (atmospheric) pressure.

54. An apparatus for the direct conversion of methane gas to methanol comprising:
i. a storage unit for methane gas;
ii. a contacting unit for passing a gas feed comprising methane gas and oxygen over an oxygen-activated catalyst according to claim 1.

55. The apparatus of claim 54 further comprising a collecting unit for removing methanol from said contacting unit.

56. The apparatus of claim 54 or 55, wherein the apparatus further comprises a heating unit for heating said oxygen-activated catalyst to a temperature of less than 750° C.
